(19) United States
(12) Patent Application Publication
(10) Pub. No.: US 2024/0023795 A1
(43) Pub. Date: Jan. 25, 2024

(54) MEDICAL DEVICE ASSEMBLIES AND COMPONENTS

(71) Applicant: Boston Scientific Scimed, Inc., Maple Grove, MN (US)

(72) Inventors: Nathan GAWORSKI, St. Paul, MN (US); Oliver J. GORST, Lowell, MA (US); John EATON, Fitchburg, MA (US); Meherdil D. DASTUR, Saint Louis Park, MN (US); James E. BLOOD, Shoreview, MN (US); Paul CARNE, Escanaba, MI (US); Bradley S. SWEHLA, Eagan, MN (US)

(73) Assignee: Boston Scientific Scimed, Inc., Maple Grove, MN (US)

(21) Appl. No.: 18/353,381

(22) Filed: Jul. 17, 2023

Related U.S. Application Data
(60) Provisional application No. 63/368,831, filed on Jul. 19, 2022.
(60) Provisional application No. 63/489,933, filed on Mar. 13, 2023.

(51) Int. Cl. A61B 1/05; A61B 1/06

(52) U.S. Cl. CPC A61B 1/051; A61B 1/0676; A61B 2562/164

(57) ABSTRACT

A flexible circuit assembly for a medical device, the circuit assembly including a circuit board having at least one bending portion and at least one flat portion, and a mechanical support structure coupled to the circuit board. The mechanical support structure includes a first region and a second region extending distally from the first region. The at least one bending portion of the circuit board is a portion of an arm, the arm extending distally from the flat portion, and the at least one bending portion of the circuit board is coupled to the second region of the mechanical support structure.

### CROSS-REFERENCE TO RELATED APPLICATIONS

This application claims priority under 35 U.S.C. § 119 from U.S. Provisional Application No. 63/489,933, filed on Mar. 13, 2023, and U.S. Provisional Application No. 63/368,831, filed on Jul. 19, 2022, the entirety of each of which is incorporated herein by reference.

### TECHNICAL FIELD

The disclosure relates generally to devices, systems, and methods for medical device assemblies and components. More specifically, aspects of the disclosure pertain to devices, systems, and/or methods that include flexible circuit boards or flexible circuit board assemblies.

### BACKGROUND

In a medical procedure, an operator may insert a medical device, such as an endoscope, or other type of scope, into a body lumen of a subject. To provide visibility to the operator during the procedure, the scope may include an imaging system disposed within a distal assembly of the scope. Such an imaging system may include an illumination element and a camera. The imaging system components may be incorporated onto a flat circuit board; however, such flat circuit boards may occupy a relatively large space in the distal assembly. Therefore, a need exists for systems, devices, and/or methods that include flexible circuit boards and/or flexible circuit board assemblies.

### SUMMARY

Each of the aspects disclosed herein may include one or more of the features described in connection with any of the other disclosed aspects.

Aspects of the disclosure relate to, among other things, systems, devices, and methods relating to flexible circuit board assemblies configured to be used within medical devices.

According to an example, a flexible circuit assembly for a medical device may include a circuit board having at least one bending portion and at least one flat portion and a mechanical support structure coupled to the circuit board. The mechanical support structure may include a first region and a second region extending distally from the first region, where the at least one bending portion of the circuit board is a portion of an arm, the arm extending distally from the flat portion, and where the at least one bending portion of the circuit board is coupled to the second region of the mechanical support structure.

Any of the flexible circuit assemblies described herein may include any of the following features. The first region includes at least one extension, where the at least one extension is configured to receive an articulation wire of the medical device. The at least one bending portion includes a first layered construction and the at least one flat portion includes a second layered construction. The first layered construction includes fewer layers than the second layered construction. The first layered construction is configured to achieve a bending radius less than 6 times a thickness of the second layered construction. The circuit board comprises at least one mounting portion, where the at least one bending portion is disposed on a proximal portion of the at least one mounting portion. The at least one mounting portion is coupled to a mounting pad. The mounting pad is configured to receive one of a camera or a lighting element. The at least one flat portion defines a first plane, where a flat portion of the arm defines a second plane parallel to the first plane. A distal portion of the arm defines a third plane, where the third plane is perpendicular to each of the first plane and the second plane. The circuit board is coupled to the mechanical support structure with an adhesive. The at least one flat portion of the circuit board is coupled to the first region of the mechanical support structure. The mechanical support structure comprises at least one of a polymeric material or a metal. The mechanical support structure comprises at least one of a thermoplastic material or a thermoset material. The flexible circuit assembly is configured to be incorporated into a distal assembly of an endoscope.

According to another example, a flexible circuit assembly for a medical device may include a circuit board having at least one arm extending distally from a flat portion of the circuit board and at least one bending portion within the at least one arm, and a mechanical support structure coupled to the circuit board. The mechanical support structure may have at least one arm extending distally from a first portion of the mechanical support structure, where the at least one arm of the mechanical support structure is configured to be coupled to the at least one arm of the circuit board.

Any of the flexible circuit assemblies described herein may include any of the following features. The at least one bending portion includes a first layered construction and the flat portion comprises a second layered construction. The first layered construction includes an adhesive layer, a conductive layer, and at least one flexible layer. The second layered construction includes an adhesive layer, a conductive layer, at least one flexible layer, and at least one dielectric layer.

According to another example, a flexible circuit assembly for a medical device may include a circuit board having a first portion and a second portion extending distally from the first portion and a mechanical support structure coupled to the circuit board. The mechanical support structure may include a first region and a second region extending distally from the first region, where the first portion includes a first layered construction and the second portion includes a second layered construction, where the second portion includes an arm including a bending portion, where the circuit board is configured to be bent at the bending portion and to be unbent at the first portion, and where the bending portion of the circuit board is coupled to a distal face of the second region of the mechanical support structure.

### BRIEF DESCRIPTION OF THE DRAWINGS

The accompanying drawings, which are incorporated in and constitute a part of this specification, illustrate examples of this disclosure and, together with the description, serve to explain the principles of the disclosure.

FIG. 1A depicts a perspective view of a flexible circuit assembly according to some embodiments.

FIG. 1B depicts a plan view of a circuit board of the flexible circuit assembly of FIG. 1A.

FIG. 2 depicts a top perspective view of the flexible circuit assembly of FIG. 1A.

FIG. 3 depicts a side perspective view of the flexible circuit assembly of FIG. 1A.

FIG. 4 depicts a perspective view of a flexible circuit assembly including a wire guide according to some embodiments.

FIG. 5 depicts a layered construction of a flexible region of a flexible circuit according to some embodiments.

FIG. 6 depicts a layered construction of a flexible circuit according to some embodiments.

FIG. 7 depicts a flexible circuit assembly including a flexible circuit having a layered construction according to some embodiments.

FIG. 8 depicts a flexible circuit in a flat configuration according to some embodiments.

FIG. 9A depicts a perspective view of a distal assembly of a medical device according to some embodiments.

FIG. 9B depicts a rear view of the distal assembly of FIG. 9A.

FIG. 10A depicts a perspective view of a distal assembly of a medical device according to some embodiments.

FIG. 10B depicts a front view of the distal assembly of FIG. 10A.

FIG. 11 depicts a bottom cross-sectional view of the distal assembly of FIG. 11A.

FIG. 12A depicts a perspective view of a distal assembly of a medical device according to some embodiments.

FIG. 12B depicts a front view of the distal assembly of FIG. 12A.

FIG. 13A depicts a proximal portion of a medical device according to some embodiments.

FIG. 13B depicts a distal portion of the medical device of FIG. 13A.

### DETAILED DESCRIPTION

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

A distal assembly of a medical device, such as an endoscope, may include a substrate (e.g., a circuit board), which may have mounted thereon elements such as imaging elements and/or lighting elements. Although the disclosure may reference an “endoscope” (or a “scope”), it will be appreciated that the disclosure also encompasses duodenoscopes, cholangioscopes, bronchoscopes, gastroscopes, endoscopic ultrasonography (“EUS”) scopes, colonoscopes, ureteroscopes, bronchoscopes, laparoscopes, cystoscopes, aspiration scopes, sheaths, catheters, or similar devices. References to an “endoscope” will be understood to include any of the above devices. Imaging elements may include one or more image sensors, cameras, or fiber optic light guides. Lighting elements may include one or more (e.g., two) light emitting diodes (“LEDs”) or fiber optic light guides. The circuit board may also have mounted thereon one or more position-sensing systems, capacitors, diodes, resistors, digitization chips, analog to digital converters, and/or additional sensors, for example force, pressure, or temperature sensors.

Circuit boards having flat configurations may be incorporated into the above-described distal assemblies. However, if the sizes of the distal assemblies are decreased, for example to accommodate a smaller body lumen or to be inserted into a working channel of another endoscope, a flat circuit board may occupy a greater amount of space in the distal assembly than desired. Smaller circuit boards may be used, but the smaller surface area may create difficulties in the manufacturing process or may prohibit inclusion of elements such as position-sensing systems, capacitors, diodes, resistors, digitization chips, analog to digital converters, and/or additional sensors, for example force, pressure, or temperature sensors. Accordingly, disclosed herein are embodiments of a flexible circuit board, which may be assembled in a flat configuration before being bent into a shape configured to fit within a distal assembly of a scope, such as an endoscope. By assembling the circuit board while it is in a flat configuration, automated, pick and place assembly, or other methods may be utilized to mount various components on the flat circuit board, thereby simplifying the manufacturing process. The circuit board may subsequently be bent and assembled within/on the distal tip assembly. Embodiments of the flexible circuit boards described herein may be incorporated into a flexible circuit assembly, which may additionally include a mechanical support structure configured to provide rigidity to the flexible circuit during manufacturing and to aid in forming the final geometry of the circuit, as well as to assist in assembly of the distal tip assembly.

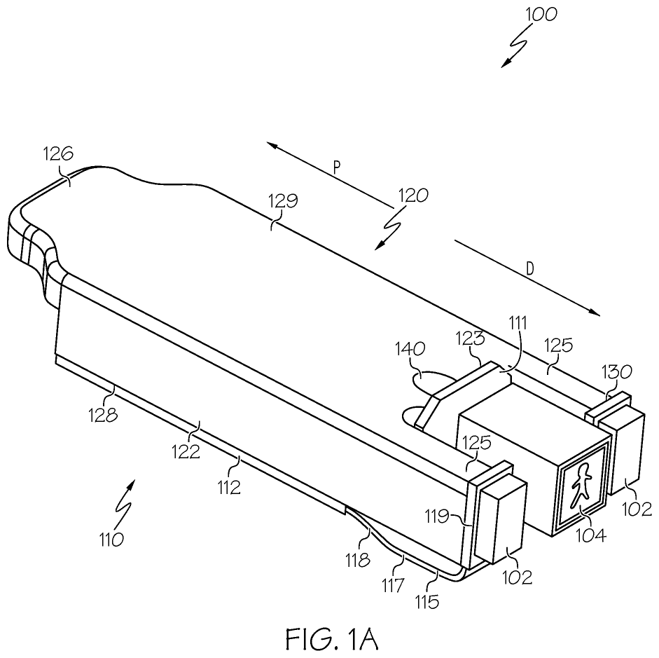

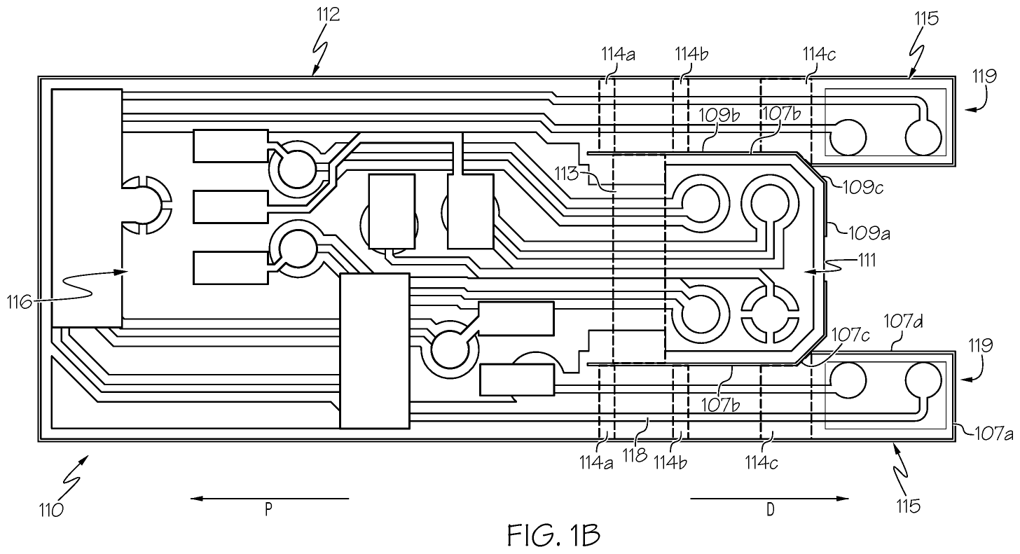

For example, FIGS. 1A-3 depict an exemplary flexible circuit assembly 100 and components thereof, according to some embodiments. A proximal direction of FIGS. 1A and 1B is indicated by the arrow “P,” and a distal direction of FIGS. 1A and 1B is indicated by the arrow “D.” In some embodiments, flexible circuit assembly 100 may include a flexible circuit 110 (which may include a flexible circuit board and/or one or more elements mounted thereon) coupled to a mechanical support structure, for example skeleton 120. FIGS. 1A, 2, and 3 depict flexible circuit 110 incorporated into flexible circuit assembly 100, with flexible circuit 110 having portions thereof bent, as described below. FIG. 1B depicts flexible circuit 110 in a flattened configuration, prior to assembly on skeleton 120. Flexible circuit 110 may include, for example, polyimide or another flexible media, such as liquid crystal polymer (“LCP”) and may or may not include a stiffener. Flexible circuit 110 may be designed with a desired (e.g., predetermined) impedance. Flexible circuit 110 may include soldermask and/or coverlay. Alternatively, flexible circuit 110 may include a via in pad construction instead of soldermask (e.g., to avoid using soldermask).

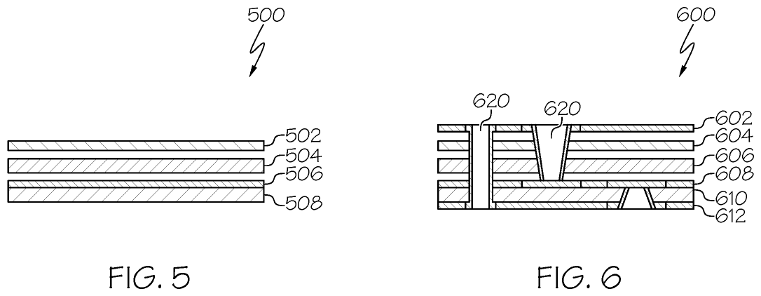

Flexible circuit 110 may include a flat (i.e., unbent) portion 112 and bending portions 113 and 114a, 114b, 114c. In some examples, an entirety of flexible circuit 110 may be flexible. Bending portions 113, 114a, 114b, 114c (FIG. 1B) may be portions of flexible circuit 110 that are bent/flexed when constructed in flexible circuit assembly 100. Bending portions 113, 114a, 114b, 114c may incorporate, for example, a layered construction 500, discussed below with respect to FIG. 5. Other portions of flexible circuit 110 (i.e. portions of flexible circuit 110 that remain flat or unbent) may incorporate a layered construction 600, discussed below with respect to FIG. 6. Positions of bending portions 113, 114a, 114b, 114c indicated in FIGS. 1A-3 are merely exemplary, and other portions of flexible circuit 110 may additionally or alternatively be bent in constructing flexible circuit assembly 100. In other examples, portions of flexible circuit 110 may be rigid, such that flexible circuit 110 is a rigid-flex circuit. For example, bending portions 113, 114a, 114b, 114c, shown in FIG. 1B, may be flexible, while other portions of flexible circuit 110 may be rigid.

In some embodiments, flexible circuit 110 may include at least one arm 115 extending distally from flat portion 112, and bending portions 114a, 114b, 114c may be incorporated into arm 115 (e.g., as shown in FIG. 1B). Arm 115 may be configured to bend at bending portions 114a, 114b, 114c to allow flexible circuit 110 to achieve a particular geometry (e.g., as shown in FIGS. 1A, 2, and 3). Positions of bending portions 114a, 114b, 114c shown in FIG. 1B are merely exemplary. Other portions of arm 115 also may be bent in constructing flexible circuit assembly 100. As shown in FIGS. 1A-3, flexible circuit 110 may include two arms 115. However, in some embodiments, flexible circuit 110 may include at least three arms. Each of arms 115 may have corresponding bending portions 114a, 114b, 114c, as shown in FIG. 1B.

Flexible circuit 110 may also have a mounting portion 111, which may extend distally from flat portion 112, between arms 115. Bending portion 113 may be disposed on a proximal portion of mounting portion 111.

Various components, for example lighting elements 102 and a camera 104 may be mounted onto flexible circuit 110. In some embodiments, lighting element(s) 102 may be connected to a distal portion 119 of each arms 115, while camera 104 may be connected to a mounting portion 111. However, in some embodiments, camera 104 and lighting element(s) 102 may all be mounted to the same portion of flexible circuit 110 (e.g., mounting portion 111 or distal portion 119).

Additional components, for example capacitors, diodes, resistors, or other sensors may be mounted to flat portion 112 and/or to mounting portion 111 or distal portion 119. Lighting elements 102 may have any suitable features and may include, for example, LEDs of any suitable size and properties. Camera 104 may have any suitable features and may include, for example a ball grid array (“BGA”)-style camera. Camera 104 may have any suitable size, any suitable shape, and any suitable image sensor/lens arrangement. Components attached to flexible circuit 110 may be attached using any suitable attachment method known in the art, for example one or more of surface mount technology, reflow, soldering, potting, and/or encapsulation. In some embodiments, any of the components described above may be included in an assembly separate from flexible circuit 110, which may be configured to work in conjunction with flexible circuit 110 or may be omitted entirely.

As shown particularly in FIG. 1A, together with FIG. 1B, when flexible circuit 110 is in a bent configuration (FIG. 1A), flat portion 112 may define a first plane. Each arm 115 may include a first flat portion 117 that defines a second plane, between bending portions 114b and 114c. When incorporated into assembly 100, the second plane of first portion 117 may be approximately parallel to the first plane of flat portion 112 but may be offset from the first plane. A ramp portion 118 may extend between flat portion 112 and first portion 117 (e.g., between bending portions 114a, 114b). Alternatively, the second plane of first portion 117 and the first plane of flat portion 112 may be coplanar. Distal portion 119 of arm 115 may be distal of bending portion 114c and may define a third plane. Lighting element 102 may be mounted to distal portion 119. The third plane of distal portion 119 may be approximately perpendicular to the first plane of flat portion 112 and/or the second plane of first portion 117.

Mounting portion 111 (extending distally from bending portion 113 in the flat configuration of FIG. 1B) may define a fourth plane that is approximately perpendicular to the first plane of flat portion 112 and/or the second plane of first portion 117 when flexible circuit 110 is in the bent configuration of FIG. 1A (i.e., when flexible circuit 110 is incorporated into flexible circuit assembly 100). The fourth plane of mounting portion 111 may be approximately parallel to the third plane of distal portion 119. The relative angles discussed above are merely exemplary, and any suitable arrangement may be utilized to achieve a desired arrangement of elements such as camera 104 and lighting elements 102. For example, relationships discussed as approximately perpendicular above may have alternative transverse angles or parallel relationships. Similarly, relationships described as approximately parallel above may have transverse relationships in alternative aspects.

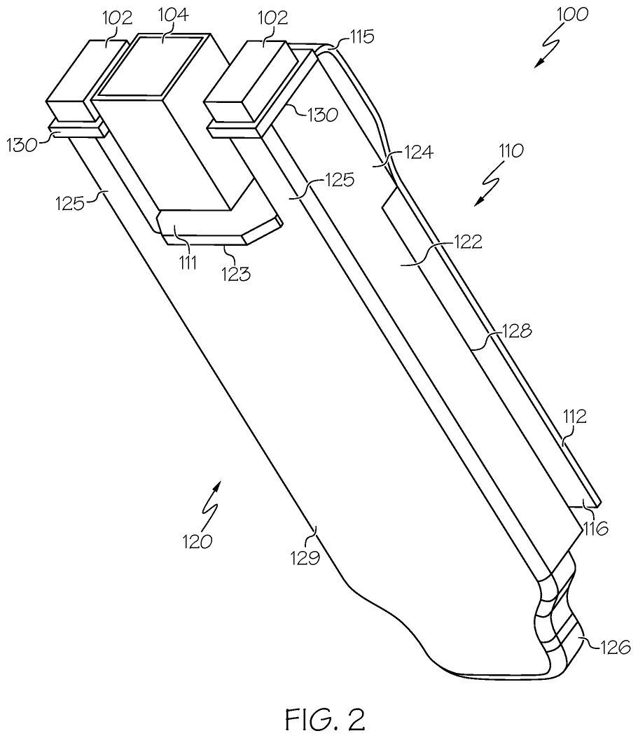

As shown in FIGS. 1A-3, mounting portion 111 may be proximal of distal portion 119 when flexible circuit 110 is in a bent configuration. A position of mounting portion 111 may accommodate a greater depth (in a proximal/distal direction) of camera 104 as compared to a depth of lighting elements 102. A distal end of camera 104 may be distal to distal ends of lighting elements 102, as shown particularly in FIG. 2. Alternatively, a distal end of camera 104 may be approximately level with distal ends of lighting elements 102 or proximal of distal ends of lighting elements 102.

In a flat configuration of flexible circuit 110, shown in FIG. 1B, arms 115 may extend distally from flat portion 112, such that outer edges of flexible circuit 110 that run in a proximal/distal direction may continue in a straight line (e.g., approximately parallel to a central longitudinal axis of flexible circuit 110 and/or a central longitudinal axis of the endoscope into which flexible circuit 110 is incorporated) along flat portion 112 and arms 115. In the flat configuration, outer lateral edges 109b of mounting portion 111 may abut or be near to proximal inner edges 107b of arms 115. A distal edge 109a of mounting portion 111 may be proximal of distal edges 107a of arms 115 in the flat configuration. Mounting portion 111 may be approximately rectangular in the flattened configuration and may have diagonal edge portions 109c extending between distal edge 109a of mounting portion 111 and lateral edges 109b of mounting portion 111. Arms 115 may each have a complementary diagonal edge portion 107c, which may abut or be near to diagonal edge portion 109c of mounting portion 111. Diagonal edge portion 107c of arm 115 may extend between proximal inner edge 107b and a distal inner edge 107d of arm 115. At portions of arm 115 defined by proximal inner edge 107b, arm 115 may be narrower (along an up/down direction of FIG. 1B) than at portions of arm 115 defined by distal inner edge 107d.

Still referring to FIGS. 1A-3, to provide support to flexible circuit 110 during manufacturing, assembly of a distal tip assembly, and use of the endoscope, skeleton 120 may be coupled to flexible circuit 110. Accordingly, in some embodiments, skeleton 120 may have a similar, complementary, or corresponding geometry to flexible circuit 110. For example, skeleton 120 may include a first region 122 configured to be coupled to or otherwise lie adjacent to flat portion 112 of flexible circuit 110, and a second region 124, extending distally from first region 122 and configured to be coupled to arm(s) 115 of flexible circuit 110. In some embodiments, skeleton 120 may solely be coupled to flexible circuit 110 via (1) a connection between second region 124 of skeleton 120 and arm 115 (e.g., distal portion 119) of flexible circuit 110 and (2) a connection between bending portion 113 and/or mounting portion 111 and a distal end 123 of first region 122, as discussed in further detail below. In other embodiments, flat portion 112 may be coupled to first region 122 (e.g., via adhesive).

In some embodiments, second region 124 may include at least one arm 125, which may mirror the geometry of arm 115 of flexible circuit 110. As shown in FIGS. 1A-3, second region 124 may include two arms 125. In some embodiments, a length of arm 125 may be selected to determine a position of lighting element 102. For example, the length of arm 125 may be shortened as compared to FIGS. 1A-3 to recess a position of lighting element 102 behind camera 104.

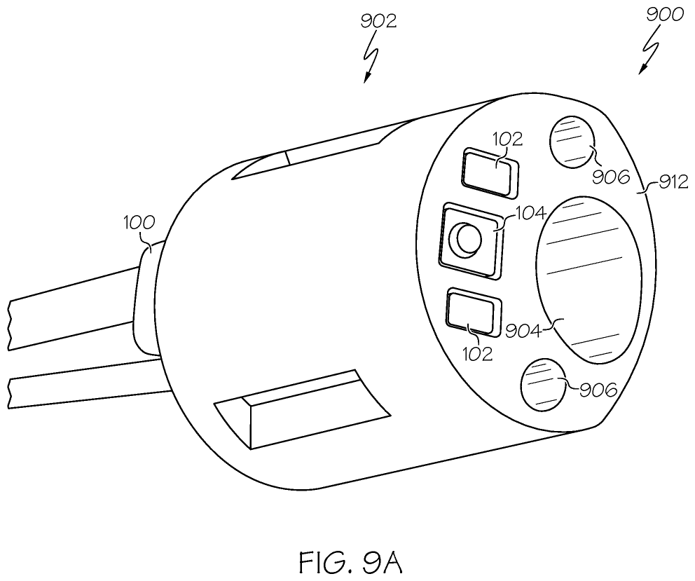

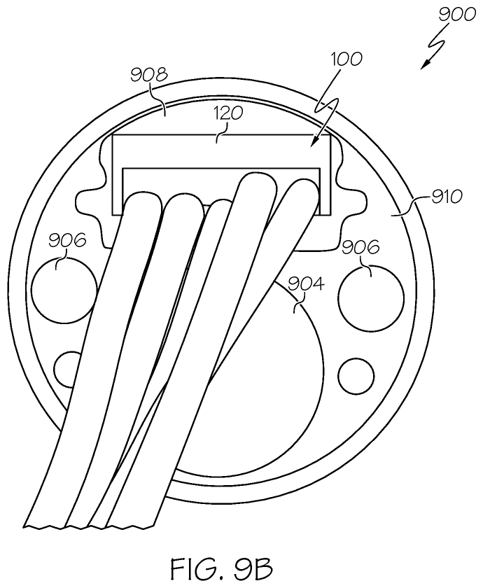

Additionally, skeleton 120 may include a third region 126, which may extend proximally from first region 122, and may function to help maintain the position of cables extending from flexible circuit 110, accommodate a shape of distal assembly housing, and/or provide a grasping point for a user to hold onto while assembling a distal assembly (FIGS. 9A and 9B). In some embodiments, third region 126 may be tapered inwardly in a lateral direction with respect to first region 122.

In some embodiments, skeleton 120 may fix flexible circuit 110 into a set position after it is bent, thereby preventing further bending both during assembly into a distal assembly of an endoscope, and during use. In other words, skeleton 120 may retain flexible circuit 110 in a desired shape/configuration.

In a bent configuration of flexible circuit 110, flat portion 112 of flexible circuit 110 may extend along a first side 128 of skeleton 120, along first region 122. First side 128 of first region 122 may have substantially the same shape as flat portion 112 (e.g., rectangular, as shown in FIGS. 1A-3). First side 128 may be substantially planar and substantially parallel to flat portion 112.

Distal portions 119 of flexible circuit 110 may be coupled to skeleton 120 at interface(s) 130. Interface(s) 130 may be between a distal face of arm 125 and a proximal surface of distal portion 119 of flexible circuit 110. A distal face of arm 125 may be approximately perpendicular to flat portion 112 of flexible circuit 110/first side 128 and approximately parallel to distal portion 119 of flexible circuit 110. Distal portions 119 may extend from first side 128 of skeleton 120, toward a second side 129 of skeleton 120. Because distal portion 119 is mounted to the distal face of arm 125 at interface 130, arm 125 may serve to retain distal portion 119 in a desired configuration (e.g., approximately perpendicular to flat portion 112).

Mounting portion 111 may be mounted to a distal end 123 of first region 122, between arms 125. As shown in FIGS. 1A-3, mounting portion 111 may occupy almost an entirety of a portion of distal end 123 of skeleton 120 that is between arms 125. As discussed above, mounting portion 111 may be retained (via being coupled to distal end 123) proximally of distal portion 119 of arms 11. Mounting portion 111 may extend from first side 128 of skeleton 120, toward second side 129 of skeleton 120. The coupling of mounting portion 111 to distal end 123 may serve to retain mounting portion 111 in a desired configuration (e.g., approximately perpendicular to flat portion 112).

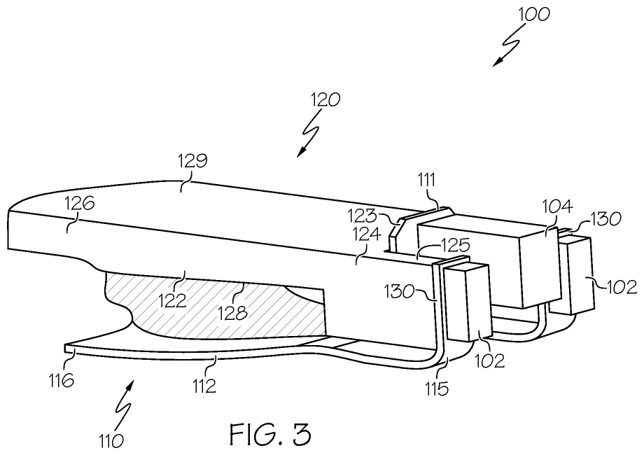

Flexible circuit 110 may be coupled to skeleton 120 by an adhesive (e.g., UV-cure adhesive). In some embodiments, adhesive 106 may be disposed between a first face 116 of flexible circuit 110 and a first side 128 of skeleton 120 (FIG. 3). Adhesive 106 may be used to couple flexible circuit 110 to skeleton 120 at interface 130, behind lighting elements 102 and/or to couple mounting portion 111 to distal end 123.

In some embodiments, skeleton 120 may include at least one depression 140 disposed in second side 129 of skeleton 120, adjacent to mounting portion 111. Depression 140 may facilitate coupling of skeleton 120 to flexible circuit 110, as adhesive may flow from second side 129 to mounting portion 111 via depression 140. Additionally, or alternatively, depression 140 may function to accommodate any components which may be mounted on a rear side of mounting portion 111.

Skeleton 120 may have any suitable shape in order to support elements of flexible circuit 110 and to accommodate a shape/configuration of the distal tip assembly of the endoscope. For example, skeleton 120 may have an approximately constant thickness between first side 128 and second side 129 at first region 122 and second region 124 of skeleton 120. Alternatively, second region 124 (including arms 125) may have a greater thickness than first region 122 (e.g., arms 125 may extend beyond first side 128). Third region 126 of skeleton 120 may have a smaller thickness between first side 128 and second side 129 or may have a same or similar thickness as first region 122 and second region 124. The above configurations are merely exemplary, and other arrangements also fall under the scope of the disclosure.

In some embodiments, skeleton 120 may be made from at least one of a polymeric material, a ceramic, or a metal. In some embodiments, skeleton 120 may be made from at least one of a thermoplastic material or a thermoset material. However, in some embodiments, skeleton 120 may be made from a thermally conductive metal, for example copper. Skeleton 120 may be manufactured through an additive manufacturing process or an injection molding process; however, any materials process technique suitable for manufacturing metal or polymeric articles may be utilized to manufacture skeleton 120.

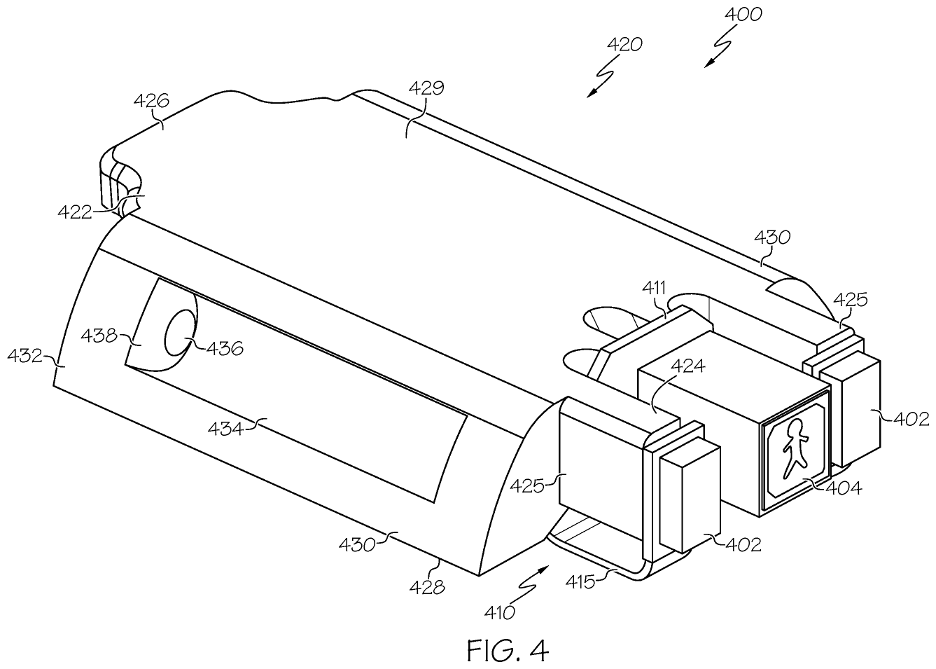

In some embodiments, for example as shown in FIG. 4, a skeleton 420 may include additional wire guiding features. Except as specified herein, a flexible circuit assembly 400 may have any of the features of flexible circuit assembly 100. Flexible circuit assembly 400 may include a flexible circuit 410 coupled to a skeleton 420. Similar to flexible circuit 110, described above, flexible circuit 410 may include a flat portion (not shown) and arms 415 extending distally from the flat portion. Arm 415 may include at least one bending portion, which may be configured to bend to allow flexible circuit 410 to achieve a particular geometry. Like flexible circuit 110, for example, components such as lighting elements 402 and camera 404 may be mounted onto flexible circuit 410. Additional components, for example capacitors, diodes, resistors, or other sensors also may be mounted to flexible circuit 410.

Still referring to FIG. 4, skeleton 420 may include features similar to skeleton 120, described above. For example, skeleton 420 may include a first region 422 configured to be adjacent to the flat portion of flexible circuit 410 along a first side 428 of skeleton 420, and a second region 424, extending distally from first region 422 and configured to be coupled to arm 415 of flexible circuit 410. In some embodiments, second region 424 may include at least one arm 425, which may mirror/complement the geometry of arm 415 of flexible circuit 410. Skeleton 420 may also include a third region 426, which may extend proximally from first region 422, and may be tapered inwardly with respect to first region 422.

Additionally, in some embodiments, skeleton 420 may include at least one extension 430 extending laterally (along a direction “A” shown in FIG. 4) from first region 422. As shown in FIG. 4, skeleton 420 may include two extensions 430. In some embodiments, extension 430 may include a chamfered or filleted upper surface 432, which may extend downwardly at an angle with respect to a second side 429 of skeleton 420. Furthermore, in some embodiments, each extension 430 may include a recess 434 within upper surface 432, which may function as a pocket to receive a mechanism, for example a steering wire mechanism. Accordingly, in some embodiments, a through hole 436, configured to receive a wire or cable, such as a steering wire, may extend through a proximal end 438 of extension 430, into recess 434. Although not shown in the view of FIG. 4, another hole may extend through a distal end of extension 430 and/or extension 430 may include other features to retain the steering wire within recess 434. Alternatively, the steering wire may be secured to extension 430 via through hole 436.

Thus, skeleton 420 may provide a distal attachment point for steering/articulation wires extending from a handle of the endoscope, through a shaft of the endoscope. Skeleton 420 may include any suitable number of attachment points for the steering/articulation wires (e.g., one for each steering/articulation wire). Alternatively, only some of the steering wires may extend to/into skeleton 420.

Referring now to FIGS. 5 and 6, in some embodiments, the disclosed flexible circuits, for example flexible circuits 110 and 410, described above, may include layered constructions in both the flat portions of the circuits and in the bending portions. For example, as shown in FIG. 5, the flexible region of a circuit may have a layered construction 500, which may include at least a first layer 502, a second layer 504, a third layer 506, and a fourth layer 508. In some embodiments, first layer 502 and fourth layer 508 may be flexible dielectric layers, while second layer 504 may be an adhesive layer, and third layer 506 may be a signal layer.

In some embodiments, layers 502-508 may each have a thickness in a range of about 0.005 mm to about 0.1 mm, including subranges. In some embodiments, first layer 502 may have a thickness of about 0.012 mm, second layer 504 may have a thickness of about 0.02 mm, third layer 506 may have a thickness of about 0.009 mm and fourth layer 508 may have a thickness of about 0.025 mm. In some embodiments, a total thickness of layered construction 500 may be in a range of about 0.02 mm to about 0.4 mm, including subranges.

In some embodiments, second layer 504 may include a polypropylene adhesive. In some embodiments, third layer 506 may be made from copper (or another conductive material), which may have the potential for trace cracking during bending, if subjected to tension or compression. Such trace cracking could potentially cause a disconnection between the flexible circuit board and any components mounted thereon when the circuit board is being bent into position. However, in some embodiments, third layer 506 may be sandwiched between first and fourth layers 502 and 508, which may each be made from a flexible material, for example a polymer such as polyimide, polyamide, or a polyester. Accordingly, during bending, third layer 506 may remain in a neutral bending axis, while first layer 502 is in compression and fourth layer 508 is in tension (or vice versa). By maintaining third layer 506 within the neutral bending axis, third layer 506 is under neither tension nor compression, and trace cracking may be significantly reduced or prevented. For example, third layer 506 may undergo at least 20 bending cycles without failure. Furthermore, due to the layered construction, the flexible circuits may be capable of achieving bending radii of less than 6 times the thickness of the circuit while maintaining continuity throughout.

In contrast, as shown in FIG. 6, in some embodiments, the flat (i.e., non-bent) portions of the disclosed flexible circuits may include a layered construction 600, which may be different from the layered construction 500 within the flex (i.e., bent) portions of the flexible circuits. For example, layered construction 600 may include a first layer 602, a second layer 604, a third layer 606, a fourth layer 608, a fifth layer 610, and a sixth layer 612. In some embodiments, first layer 602, fourth layer 608, and sixth layer 612 may each be signal layers. In some embodiments, second layer 604 and fifth layer 610 may be dielectric layers, and in some embodiments, third layer 606 may be an adhesive layer. The layers of layered construction 600 may have the same or similar materials to the corresponding layers of layered construction 500, discussed above. For example, first layer 602, fourth layer 608, and sixth layer 612 may include any of the materials of third layer 506. Second layer 604 and fifth layer 610 may include any of the materials of first layer 502 and fourth layer 508. Third layer 606 may include any of the materials of second layer 504. Additionally, in examples in which portions of a circuit are rigid, second layer 604 and fifth layer 610 (dielectric layers) of the rigid portions may include, for example, a glass-reinforced epoxy laminate such as FR-4 (flame retardant 4), a synthetic resin bonded paper such as FR-2 (flame resistant 2), a composite epoxy material (“CEM”) such as CEM-1 or CEM-3, or any combination thereof. Alternative materials also may be used within the scope of the disclosure.

In some embodiments, layered construction 600 may additionally include at least one via 620, which may be configured to transfer electrical traces to the different layers 602-610 within layered construction 600. As discussed above, layered construction 600 may also include a solder mask layer (which may include a solder resist material) adjacent to a side of first layer 602 that is opposite second layer 604. Layered construction 600 may also have such a solder mask layer adjacent to a side of sixth layer 612 that is opposite to fifth layer 610. An overlay/coverlay be adjacent to one or more of the solder mask layers (e.g., adjacent to a side of the solder mask layer that is opposite to first layer 602 or sixth layer 612).

In some embodiments, layers 602-612 may each have a thickness in a range of about 0.005 mm to about 0.1 mm, including subranges. In some embodiments, first layer 602 may have a thickness of about 0.009 mm, second layer 604 may have a thickness of about 0.012 mm, third layer 606 may have a thickness of about 0.02 mm, fourth layer 608 may have a thickness of about 0.009 mm, fifth layer 610 may have a thickness of about 0.025 mm, and sixth layer 612 may have a thickness of about 0.009 mm. In some embodiments, a total thickness of layered construction 600 may be in a range of about 0.03 mm to about 0.6 mm, including subranges.

In some embodiments, layered construction 600 may include an additional or alternative dielectric layer disposed adjacent to either, or both of, first layer 602 and fourth layer 608. In some embodiments, the additional dielectric layer may be a polymeric material or a glass epoxy. The inclusion of an additional or alternative dielectric layer in layered construction 600 may provide additional rigidity, thereby reducing the ability of the circuit board to bend in areas where layered construction 600 is incorporated.

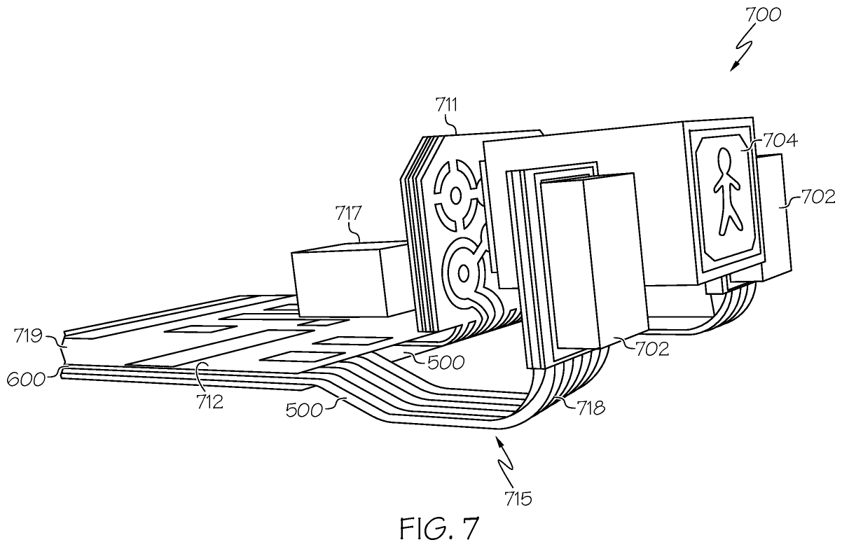

Referring now to FIG. 7, in some embodiments, layered construction 500 may be incorporated into layered construction 600. For example, in some embodiments, a flexible circuit assembly 700 (which may have any of the properties of flexible circuit assemblies 100, 200) may include a flexible circuit 710 having a flat portion 712, arms 715, and a mounting portion 711. Arms 715 may have any of the properties of arms 115, 415 described above and may have lighting elements 702 mounted thereon. Although not explicitly shown in FIG. 7, arms 715 may include a plurality of bending portions, having any of the properties and/or positions of bending portions 114a, 114b, 114c (FIG. 1B). Mounting portion 711 may have any of the properties of mounting portions 111, 411, and may have a camera 704 mounted thereon. Although not explicitly shown in FIG. 7, mounting portion 711 may include a bending portion having any of the properties and/or positions of bending portion 113 (FIG. 1B).

In some embodiments, bending portions of arms 715 and mounting portion 711 may each include a layered construction having four layers, such as layered construction 500, described above. Flat portion 712 and portions of arms 715 and mounting portion 711 that are not bent (i.e., are not bending portions) may include a layered construction having six layers, for example layered construction 600, described above. In some embodiments, layered construction 500 may define the four innermost layers of layered construction 600. In other words, layered construction 600 may add additional outer layers 602, 612 to layered construction 500. First layer 502 of layered construction 500 may correspond to second layer 604 of layered construction 600, second layer 504 of layered construction 500 may correspond to third layer 606 of layered construction 600, third layer 506 of layered construction 500 may correspond to fourth layer 608 of layered construction 600, and fourth layer 508 of layered construction 500 may correspond to fifth layer 610 of layered construction 600.

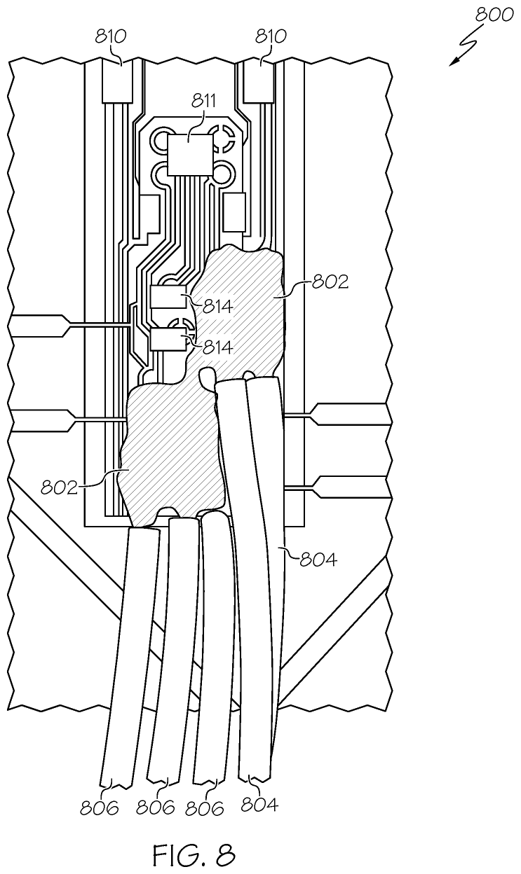

When flexible circuit assembly 700 is in a flat configuration (similar to that shown in FIG. 1B and FIG. 8, which is described in detail below), lighting elements 702 and camera 704 may be disposed on a first side 718 of flexible circuit 710. An electronic component 717 (which may be any type of electronic component, including the examples described above with respect to FIGS. 1A-3) may be disposed on a second side 719 of flexible circuit 710. Lighting elements 102, camera 104, and electronic component 717 may be placed on flexible circuit 710 using any suitable automated or manual method. Bending portions of arms 715 and mounting portion 711 may subsequently be bent into the configurations described with respect to FIGS. 1A-3, by manual or automatic methods, and may optionally be coupled to a skeleton, such as skeleton 120.

As described above, various components may be mounted to any of the flexible circuit boards described herein. As shown in FIG. 8, for example, flexible circuit 800 have any of the features of flexible circuits 110, 410, 710, and may include several pads to which components may be mounted. For example, flexible circuit 800 may include at least one lighting element mounting pad 810, at least one camera mounting pad 811, and at least one capacitor pad 814. However, flexible circuit 800 may include more or fewer mounting pads, depending on the intended application of the circuit. Additionally, in some embodiments, lighting element cables 804 and camera cables 806 may be coupled to flexible circuit 800 with an adhesive 802. Lighting element cables 804 and/or camera cables 806 may include, for example, any combination of coaxial cable (e.g., of matched impedance) and/or ribbon cables. Lighting element cables 804 and/or camera cables 806 may have any suitable type of insulation and/or conductor. In alternative to cables (e.g., lighting element cables 804 and camera cables 806), a long-format flexible circuit board may be utilized.

Still referring to FIG. 8, all of the above-described components may be mounted onto flexible circuit 800 while flexible circuit 800 is in a flat configuration. Subsequently, circuit 800 may be coupled with a skeleton, for example any of the skeletons described herein, and may be bent into a bent configuration (such as those shown in FIGS. 1A, 2, 3, 4, and/or 7) prior to being inserted into a distal assembly of an endoscope.

As shown in FIGS. 9A and 9B, once the flexible circuit assemblies are assembled, they may be incorporated into a distal assembly of an endoscope. For example, in some embodiments, flexible circuit assembly 100 may be in incorporated into distal assembly 900. Distal assembly 900 may include a body 902 having at least one irrigation channel 906 and at least one working channel 904 extending therethrough. As shown in FIG. 9B, flexible circuit assembly 100 may be inserted into a cavity 908 extending distally into body 902 from a proximal face 910 of body 902. As shown in FIG. 9A, when flexible circuit assembly 100 is fully inserted into body 902, lighting elements 102 and camera 104 may be flush with, or slightly recessed relative to, a distal face 912 of body 902.

In some embodiments, the rigidity provided by skeleton 120, described above, may aid in inserting flexible circuit assembly 100 into cavity 908, and may additionally facilitate the process of adhering or otherwise securing flexible circuit assembly 100 to body 902. For example, skeleton 120 may provide a large surface area on which an adhesive may be applied. Alternatively, in some embodiments skeleton 120 may simply be cast into place (e.g., encapsulated) within cavity 908.

Additionally, cavity 908 may have a geometry corresponding to a geometry of skeleton 120 to help ensure that skeleton 120 fits snugly within body 902. Accordingly, cavity 908 may have a width and a height corresponding to a width and a height of skeleton 120, respectively and/or elements positioned on flexible circuit assembly 100. However, the geometry of cavity 908 may include any shape or configuration suitable to receive a circuit assembly.

In some embodiments, alternative flexible circuit assemblies may be incorporated into a distal assembly of an endoscope. For example, as shown in FIGS. a flexible circuit assembly 1100 may be incorporated into a distal assembly 1000. Similar to distal assembly 900, described above, distal assembly 1000 may include a body 1002 having at least one irrigation channel 1006 and at least one working channel 1004 extending therethrough.

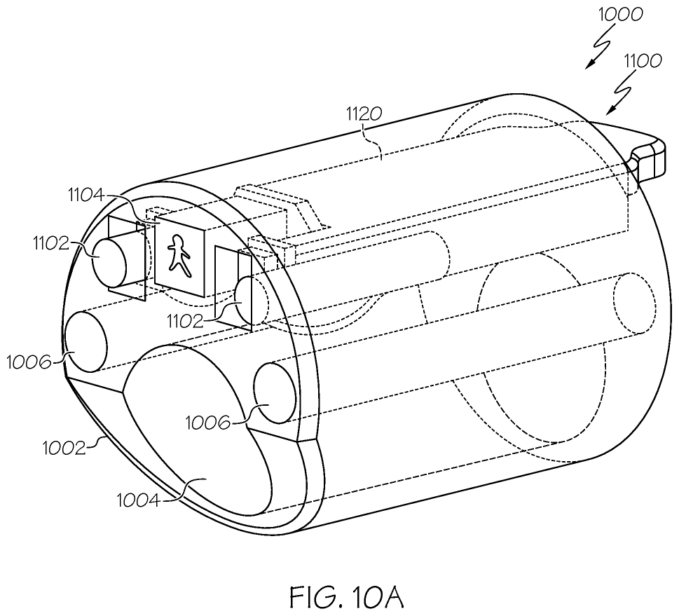

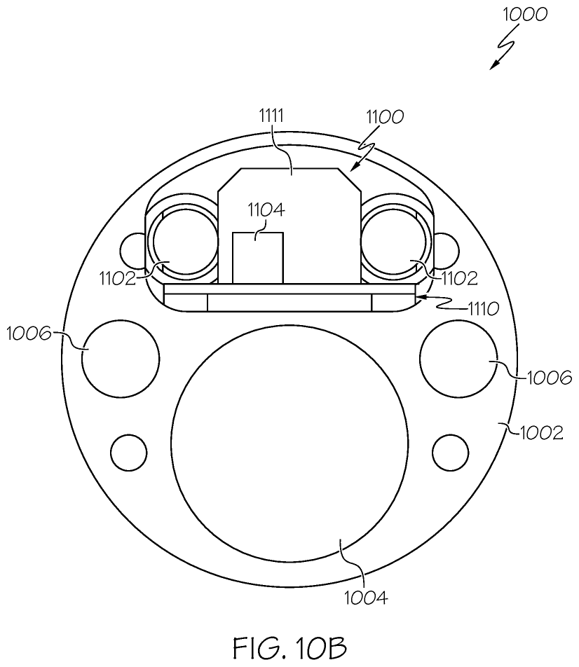

Flexible circuit assembly 1100 may include a skeleton 1120, similar to skeletons 120 and 420, discussed above; however, at least one optical fiber 1102, such as a plastic optical fiber, may be coupled to skeleton 1120 or extend adjacent to skeleton 1120, in place of a lighting element mounted to a flexible circuit. As shown in FIGS. 10A and 10B, optical fiber(s) 1102 may be positioned along either side of skeleton 1120, adjacent to flexible circuit 1110 (FIG. 10B). Accordingly, skeleton 1120 may be formed without an arm portion, as flexible circuit 1110 may not include an arm portion on which a lighting element may be mounted. Rather, as shown in FIG. 10B, flexible circuit 1110 may solely include a mounting portion 1111 to which a camera 1104 may be mounted. In some embodiments, optical fibers 1102 may extend adjacent to mounting portion 1111 on either side of mounting portion 1111. For example, a first optical fiber 1102 may extend adjacent to mounting portion 1111 on a first side of mounting portion 1111, and a second optical fiber 1102 may extend adjacent to mounting portion 1111 on a second side of mounting portion 1111, opposite the first side. A surface of flexible circuit 1110 other than mounting portion 1111 may support or otherwise be adjacent to optical fibers 1102 and may assist in positioning optical fibers 1102 in a desired position.

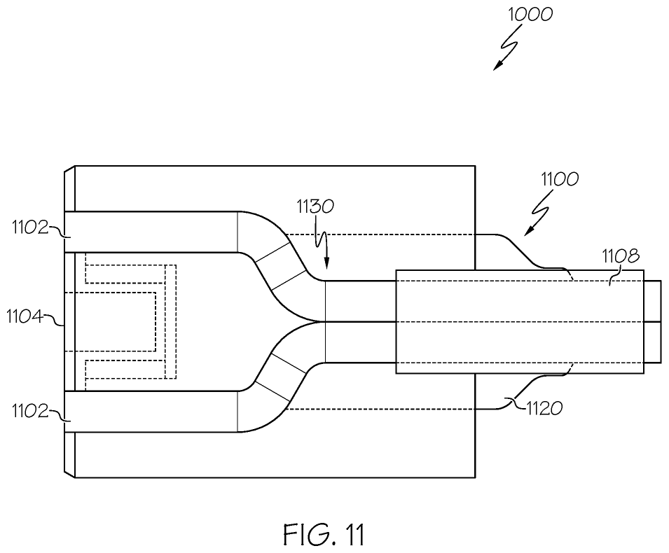

As shown in FIG. 11, for example, optical fibers 1102 may be mounted to flexible circuit 1110 in a y-shaped configuration such that each of optical fibers 1102 extends directly adjacent to one another from a proximal end of flexible circuit assembly 1000 until they reach a junction 1130. At junction 1130, each of optical fibers 1102 may extend away from one another at an angle before then extending parallel to the longitudinal axis of flexible circuit assembly 1000 and adjacent mounting portion 1111. As shown in FIG. 11, each optical fiber 1102 may include a first segment, a second segment, and a third segment. The first segment and the third segment may be substantially parallel to one another and/or to the longitudinal axis of flexible circuit assembly 1000. The second segment may extend between the first segment and the third segment and may be curved or otherwise non-parallel to the first segment and the third segment. Alternatively, the segments may have other angles with respect to one another.

In some embodiments, optical fiber(s) 1102 may optionally be coupled to skeleton 1120 via a sheath 1108. Alternatively, optical fiber(s) 1102 may be coupled to skeleton 1120 using an adhesive, may be molded to skeleton 1120, or may be pressed into skeleton 1120 using mechanical interference.

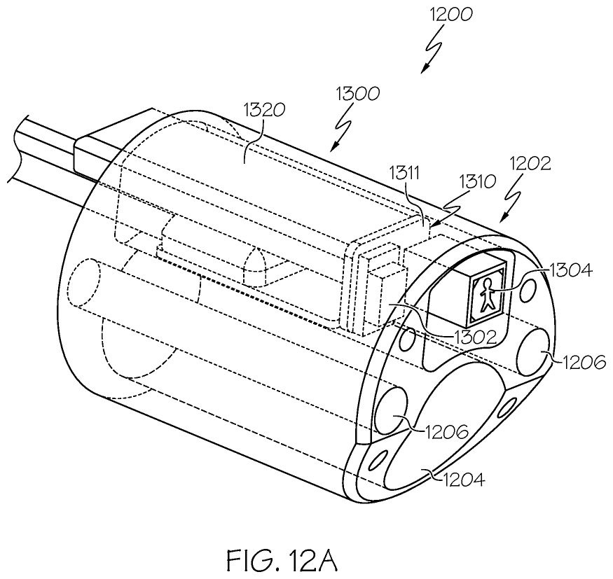

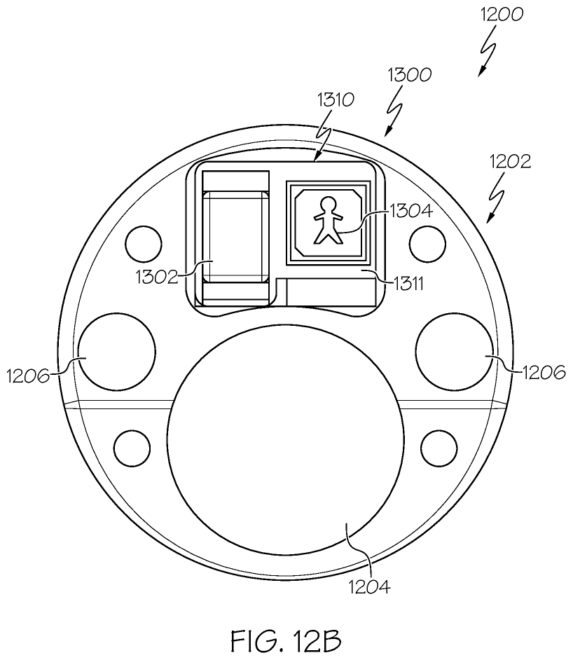

Alternatively, for example as shown in FIGS. 12A and 12B, a flexible circuit assembly 1300, including only one lighting element 1302, may be incorporated into a distal assembly 1200. Similar to distal assemblies 900 and 1000, described above, distal assembly 1200 may include a body 1202 having at least one irrigation channel 1206 and at least one working channel 1204 extending therethrough. As shown in FIG. 12B, a flexible circuit 1310 may be coupled to a skeleton 1320, and may include a mounting portion 1311 configured to receive both a camera 1304 and a single lighting element 1302. Because mounting portion 1311 may accommodate both camera 1304 and lighting element 1302, flexible circuit 1310 and skeleton 1320 may both be formed without arm portions. Accordingly, a width of flexible circuit assembly 1300 may be minimized, which may facilitate insertion of flexible circuit assembly 1300 into distal assembly 1200.

In some embodiments, for example as shown in FIG. 12A, lighting element 1302 may be positioned proximally with respect to camera 1304. However, in some embodiments, lighting element 1302 may be positioned adjacent camera 1304 such that lighting element 1302 and camera 1304 are flush with one another. Alternatively, lighting element 1302 may be configured to extend distally past camera 1304.

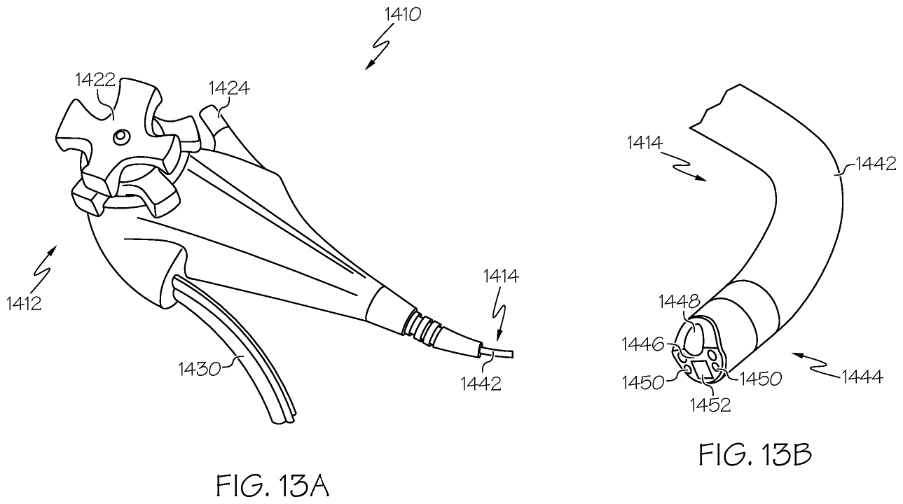

FIGS. 13A and 13B depict aspects of an exemplary medical device 1410. The assemblies discussed above may be incorporated into medical device 1410. FIG. 13A depicts a proximal portion of medical device 1410. FIG. 13B depicts a distal tip 1444 of medical device 1410, which may incorporate an assembly such as any of distal assemblies 900, 1000, or 1200. Medical device 1410 may include a handle portion 1412 for gripping and operation by an operator, and an insertion portion 1414 for at least partial insertion into a body (e.g., a body lumen) of a subject. As shown in FIGS. 13A and 13B, medical device 1410 may include an endoscope.

Handle portion 1412 may include a knob 1422, for example, on a proximal portion of handle portion 1412. Knob 1422 may help to facilitate articulation/steering of insertion portion 1414, including distal tip 1444. Although knob 1422 is depicted in FIG. 13A, it will be appreciated that any suitable actuator(s) may be used in addition to or in place of knob 1422, such as one or more knobs, buttons, sliders, or joysticks. A port 1424 of handle portion 1412 may provide access to a lumen or working channel of medical device 1410. An operator may insert an instrument or other device into port 1424 and may extend the instrument or other device distally through the working channel. The working channel may extend longitudinally through a length of insertion portion 1414. An umbilicus 1430 may extend from handle portion 1412 (e.g., from a proximal portion of handle portion 1412) and may carry wires, cables, and/or conduits for providing, for example, power, signals, or fluids to or from handle portion 1412. For example, umbilicus 1430 may connect handle portion 1412 to one or more user interfaces, monitors, displays, etc.

Insertion portion 1414 may include a shaft 1442 extending distally from handle portion 1412. Shaft 1442 may have any suitable properties. For example, shaft 1442 may be flexible and may have wires, tubes, or other features passing therethrough. Distal tip 1444 of medical device 1410, depicted in FIG. 13B, may be disposed at a distal end of shaft 1442. As shown in FIG. 13B, distal tip 1444 may include a distalmost face 1446. Distalmost face 1446 may define a working channel opening 1448. The working channel may extend between port 1424 and working channel opening 1448, such that instruments or other devices may be passed through port 1424, through the working channel, and out of working channel opening 1448. An instrument extending distally of working channel opening 1448 may be used to perform a medical procedure on a subject.

Distal tip 1444 may also include imaging components, such as one or more lighting elements 1450 and a camera 1452 (which may have any of the properties of the lighting elements and cameras disclosed above). Although two lighting elements 1450 and one camera 1452 are depicted in FIG. 13B, it will be appreciated that alternative numbers of lighting elements 1450 and camera 1452 may be utilized. Alternatively, lighting elements 1450 and camera 1452 may be combined into a single device. Lighting elements 1450 may include LEDs or any suitable alternative light source. Camera 1452 may be configured to take video and/or still images. Camera 1452 may provide a signal to a monitor (not shown), so that an operator may view a visual image provided by camera 1452 while navigating medical device 1010 through a body of a subject.

As depicted in FIG. 13B and described above, medical device 1410 may be “forward-facing.” In other words, features of distal tip 1444 (e.g., working channel opening 1448, lighting elements 1450, and camera 1452) may face distally (i.e., forward of distalmost face 1446. This disclosure also encompasses other configurations of distal tip 1044. For example, medical device 1410 may be “side-facing.” In a side-facing embodiment, working channel opening 1448, lighting elements 1450, and/or camera 1452 may be disposed on a radially outer side of distal tip 1444, so that they point in a radially outward direction, approximately perpendicularly to a longitudinal axis of insertion portion 1414.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

What is claimed is:

1. A flexible circuit assembly for a medical device, the circuit assembly comprising:
a circuit board having at least one bending portion and at least one flat portion; and
a mechanical support structure coupled to the circuit board, the mechanical support structure including:
a first region, and
a second region extending distally from the first region,
wherein the at least one bending portion of the circuit board is a portion of an arm, the arm extending distally from the flat portion, and
wherein the at least one bending portion of the circuit board is coupled to the second region of the mechanical support structure.

2. The flexible circuit assembly of claim 1, wherein the first region comprises at least one extension, and wherein the at least one extension is configured to receive an articulation wire of the medical device.

3. The flexible circuit assembly of claim 1, wherein the at least one bending portion comprises a first layered construction and the at least one flat portion comprises a second layered construction.

4. The flexible circuit assembly of claim 3, wherein the first layered construction includes fewer layers than the second layered construction.

5. The flexible circuit assembly of claim 3, wherein the first layered construction is configured to achieve a bending radius less than 6 times a thickness of the second layered construction.

6. The flexible circuit assembly of claim 1, wherein the circuit board comprises at least one mounting portion, and wherein the at least one bending portion is disposed on a proximal portion of the at least one mounting portion.

7. The flexible circuit assembly of claim 6, wherein the at least one mounting portion is coupled to a mounting pad.

8. The flexible circuit assembly of claim 7, wherein the mounting pad is configured to receive one of a camera or a lighting element.

9. The flexible circuit assembly of claim 1, wherein the at least one flat portion defines a first plane, and wherein a flat portion of the arm defines a second plane parallel to the first plane.

10. The flexible circuit assembly of claim 9, wherein a distal portion of the arm defines a third plane, and wherein the third plane is perpendicular to each of the first plane and the second plane.

11. The flexible circuit assembly of claim 1, wherein the circuit board is coupled to the mechanical support structure with an adhesive.

12. The flexible circuit assembly of claim 1, wherein the at least one flat portion of the circuit board is coupled to the first region of the mechanical support structure.

13. The flexible circuit assembly of claim 1, wherein the mechanical support structure comprises at least one of a polymeric material or a metal.

14. The flexible circuit assembly of claim 1, wherein the mechanical support structure comprises at least one of a thermoplastic material or a thermoset material.

15. The flexible circuit assembly of claim 1, wherein the flexible circuit assembly is configured to be incorporated into a distal assembly of an endoscope.

16. A flexible circuit assembly for a medical device, comprising:
a circuit board having at least one arm extending distally from a flat portion of the circuit board and at least one bending portion within the at least one arm; and
a mechanical support structure coupled to the circuit board, the mechanical support structure having at least one arm extending distally from a first portion of the mechanical support structure,
wherein the at least one arm of the mechanical support structure is configured to be coupled to the at least one arm of the circuit board.

17. The flexible circuit assembly of claim 16, wherein the at least one bending portion comprises a first layered construction and the flat portion comprises a second layered construction.

18. The flexible circuit assembly of claim 17, wherein the first layered construction includes an adhesive layer, a conductive layer, and at least one flexible layer.

19. The flexible circuit assembly of claim 17, wherein the second layered construction includes an adhesive layer, a conductive layer, at least one flexible layer, and at least one dielectric layer.

20. A flexible circuit assembly for a medical device, comprising:
a circuit board having a first portion and a second portion extending distally from the first portion; and
a mechanical support structure coupled to the circuit board, the mechanical support structure including:
a first region, and
a second region extending distally from the first region,
wherein the first portion comprises a first layered construction and the second portion comprises a second layered construction,
wherein the second portion comprises an arm including a bending portion,
wherein the circuit board is configured to be bent at the bending portion and to be unbent at the first portion, and
wherein the bending portion of the circuit board is coupled to a distal face of the second region of the mechanical support structure.
